(19) United States
(12) Patent Application Publication
(10) Pub. No.: US 2025/0040804 A1
(43) Pub. Date: Feb. 6, 2025

(54) METHOD AND DEVICE FOR OBJECTIVELY DETERMINING THE OPTIMAL CORRECTION OF AN OPHTHALMIC REFRACTION OF A SUBJECT

(71) Applicant: Essilor International, Charenton-Le-Pont (FR)

(72) Inventors: Elisa TARTAGLIA, Paris (FR); Guillaume GIRAUDET, Orleans (FR); Yannis CHENGUITI, Aubervilliers (FR)

(73) Assignee: Essilor International, Charenton-Le-Pont (FR)

(21) Appl. No.: 18/564,020

(22) PCT Filed: May 24, 2022
(86) PCT No.: PCT/EP2022/064116

§ 371 Date: Nov. 24, 2023

(30) Foreign Application Priority Data
May 25, 2021 (EP) 21305678.1

(51) Int. Cl. A61B 3/103; A61B 5/00; A61B 5/372; A61B 5/378

(52) U.S. Cl. CPC A61B 3/103; A61B 5/372; A61B 5/378; A61B 5/7267

(57) ABSTRACT

A method for objectively determining the optimal correction of an optical refraction of a subject, comprising the steps of: a) providing said eye of the subject with successive distinct lens powers and recording the corresponding successive neural signals of the subject while said eye of the subject receives a visual stimulus through each lens power, until the recorded neural signal corresponding to one of the lens power shows a maximum neural activity as compared to all the other recorded neural signals (blocks E2 to E5), b) determining that the optimal correction of the ophthalmic refraction of the subject corresponds to a lens power with which the subject exhibits a neural signal showing a given reduced neural activity as compared to the maximum neural activity obtained in step a) (blocks E6 and E7).

### TECHNICAL FIELD OF THE INVENTION

The invention relates to the field of ophthalmic refraction of a subject. More precisely the invention relates to a device and a method for determining the optimal correction of the ophthalmic refraction of a subject.

### BACKGROUND INFORMATION AND PRIOR ART

Refraction is the phenomenon that causes a light ray to change direction when it hits the interface between two different media. Refraction notably happens when a light ray hits an eye of a subject, in which case it is called “ophthalmic refraction”. Such ophthalmic refraction will be called “refraction” in the rest of the description. Said refraction can be faulty, therefore causing the images seen by the eye to form outside of the retina. Being able to measure the refraction of each eye of a subject is a key point in an ophthalmic examination, in particular when a visual correction is necessary. Today, this measure can be obtained from two different methods: objective refractive examination and subjective refractive examination.

The objective refractive examination, which does not rely on the subject's response, can be obtained using instruments currently widely available in prescribers, such as retinoscope, automatic refractometers, aberrometers, etc . . . . The objective refractive examination is performed quickly and reliably. However, the objective refractive examination provides information relative to the refractive error of the eye optics at a given instant only, such information being insufficient to determine the subject's visual needs. The objective refractive examination therefore only gives a rough estimate of the optimal correction needed by the subject.

The subjective refractive examination attempts to assess the visual function beyond the mere eye optics, by considering also the brain processes involved. It relies on the subject's response in order to determine the optimal correction giving the best possible visual acuity in far vision with minimal accommodative effort. Hence, subjective refractive examination is more effective than objective refractive examination in catching real subject's visual needs, although it is recommended to use the results of the objective refractive examination (that is to say to use the rough estimate of the optimal correction) as a starting point for the subjective refractive examination.

However, there are several factors that make subjective refractive examination not an ideal measure. Indeed, given the nature of the measurement, which is based on the subject's response only, its results can be quite noisy, as they involve information processed by several cortical areas simultaneously: areas which are important for assessing the quality of vision (or visual acuity), but also many other associated areas, not directly involved in visual perception, but which intervene in different ways depending on the subject (in terms of types of associated areas activated and level of activation), as for example decision making related areas. Such a complex and noisy process often results in the subject himself being unable to judge with confidence the correction that gives the clearest image perception. Moreover, subjects are well aware of the essential role played by the eye examination and pay particular attention to these factors which can lead to false results, or make the eye examination a stressful experience.

All these factors inevitably lead to a longer and more complex subjective refractive examination procedure. Moreover, with the subjective refractive examination, there is a great inter-operator variability, that is to say that the optimal correction determined with the subjective refractive examination is very dependent on the skills of the eye care professional who performs the examination.

Therefore, there is a need for improving the determination of the optimal correction of the refraction of a subject.

### SUMMARY OF THE INVENTION

One object of the invention is to provide a method for objectively determining the optimal correction of an ophthalmic refraction of a subject that leads to the identification of the lens power yielding to the best perception by the subject, said best perception being as comfortable as possible for the subject in the long term.

The above object is achieved by providing the method of claim 1.

In the present invention, the “ophthalmic refraction” is considered to be the refraction of one eye of the subject. The “optimal correction” of the refraction of a subject is the correction applied to the eye of the subject which allows the subject to have a clear vision in the far distance, without accommodating his eye, or with as little accommodation as possible. The optimal correction allowing this clear vision in the far distance without accommodation or as little accommodation as possible is also comfortable in the long term.

The method of the invention relies on neural activity to identify which lens power yields to the best refraction of the eye of the subject and allows the subject to feel comfortable in the long term.

Surprisingly, it appears that the neural activity of the subject in the area of the brain associated with the quality of visual perception (also called visual acuity) is maximum not when the subject does not need any accommodation (or as little accommodation as possible) to clearly see the visual stimulus but when the subject starts to accommodate. By “start to accommodate”, it is meant that the subject accommodates in a way that is measurable by the phoropter used for the examination, for instance accommodates more than 0.25 D, or more than 0.50 D or more than 1 D, depending on the accuracy of the phoropter used for the examination. Indeed, contrary to a preconceived idea, the neural activity reaches a maximum due to neural artifacts that appear in the brain of the subject and that are related to a start of accommodation response of the eye receiving a visual stimulus. Such accommodation gives the impression of an improved visual acuity for the subject but, in reality, is not necessary for the eye of the subject to see with a satisfying quality. Moreover, such accommodation may be uncomfortable in the long term for the subject. The accommodation inducing neural artifacts is for instance linked with miosis (which is a constriction of the pupil of the eye), convergence (which is the simultaneous inward movement of both eyes toward each other), or power change in the crystalline lens.

Remarkably, to determine the optimal correction, the method of the invention finds the neural signal of the subject that exhibits a given reduced neural activity as compared to the maximum neural activity of the subject that is recorded during the examination, in the area of the brain associated with visual acuity. By doing so, the method of the invention takes into account the existence of the neural artifacts induced by the start of accommodation when determining the optimal correction of refraction.

Thanks to the method of the invention, the determination of the optimal correction of the refraction of the subject is improved, while still being objective. The subject does not need to answer any question for the optimal correction to be determined, said optimal correction being determined only based on the respective neural signals recorded when at least one eye of the subject receives a visual stimulus through respective lens powers.

According to an advantageous embodiment of the method of the invention, in step b), the given reduced neural activity is determined by correcting the maximum neural activity based on a predetermined factor k that is associated with the start of accommodation response of the eye.

More precisely, in this advantageous embodiment, factor k takes into account and removes the neural artifacts induced by the start of accommodation that may appear while the optimal correction is being searched. Thanks to factor k, the determination of the optimal correction of refraction of the subject is improved, while still being objective.

According to another embodiment, in step b), the given reduced neural activity is determined by machine learning, taking into account sets of neural signals that were recorded for many subjects whose eyes were provided with successive distinct lens powers and whose optimal correction is already precisely known.

Other advantageous characteristics of the method of this invention, taken together or separately, are given in claims 3 to 12.

A further object of the invention is to provide a device for objectively determining the optimal correction of the ophthalmic refraction of a subject that leads to the identification of the lens correction yielding to the best perception of the subject, said best perception being as comfortable as possible for the subject in the long term.

The above object is achieved by providing the device of claim 13.

The device of the invention more precisely comprises at least a neuro-sensor, for example electrodes, that is able to detect a neural signal originating from at least one area of the brain of the subject linked to visual acuity, that is to say the quality of the visual perception of the eyes of the subject, and a control unit that records the respective neural signals obtained when the eye of the subject receives a visual stimulus through respective lens powers, finds the recorded neural signal that is associated with the maximal neural activity and determines which neural signal would show the given reduced neural activity as compared to the maximum neural activity obtained in step a). The control unit then finds which lens power would give such neural signal of reduced neural activity and concludes that this lens power is the optimal correction of the refraction of the subject.

Advantageously, the device of the invention allows an objective determination of the optimal correction of the refraction of the subject, independently of the operator of the device, and without asking any question to the subject.

Other advantageous characteristics of the device of this invention, taken together or separately, are given in claims 13 and 14.

Advantageously, the device comprises at least one active power lens whose power is driven by the control unit for providing said distinct lens powers through which the eye of the subject receives the visual stimulus depending on the analysis of neural activity of the previously recorded neural signal(s).

According to an embodiment, the at least one active power lens is embedded in an eyeglass or in a contact lens.

### DETAILED DESCRIPTION OF EXAMPLE(S)

The following description with reference to the accompanying drawings will make it clear what the invention consists of and how it can be achieved. The invention is not limited to the embodiment/s illustrated in the drawings. Accordingly, it should be understood that where features mentioned in the claims are followed by reference signs, such signs are included solely for the purpose of enhancing the intelligibility of the claims and are in no way limiting on the scope of the claims.

In the accompanying drawings:

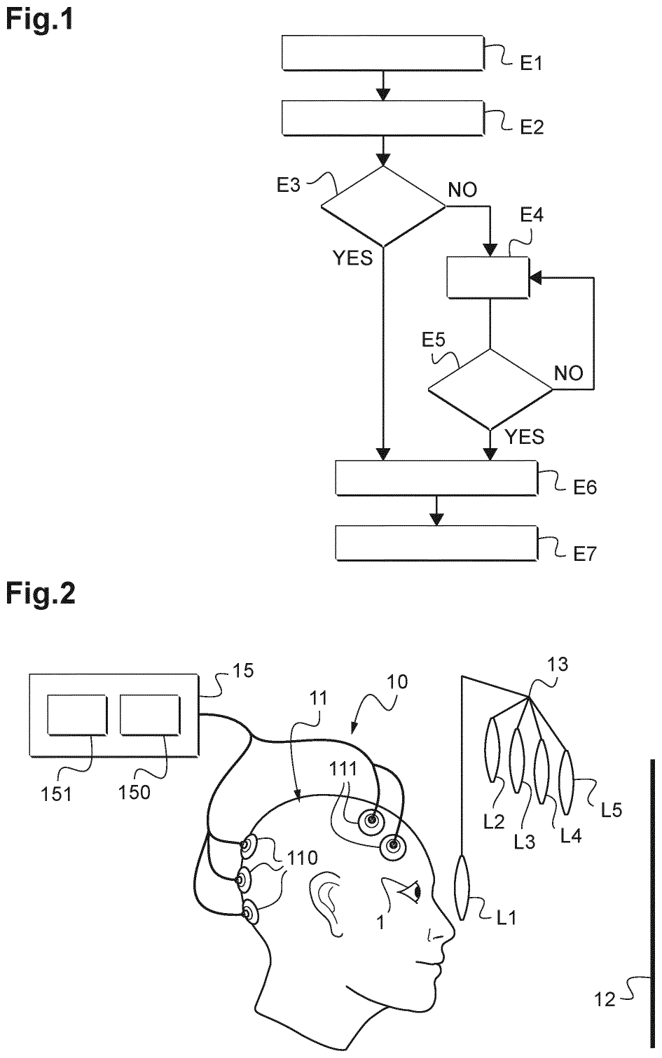

FIG. 1 is a flow chart synthetizing the main steps of the method of the invention,

FIG. 2 is a schematic representation of the device of the invention,

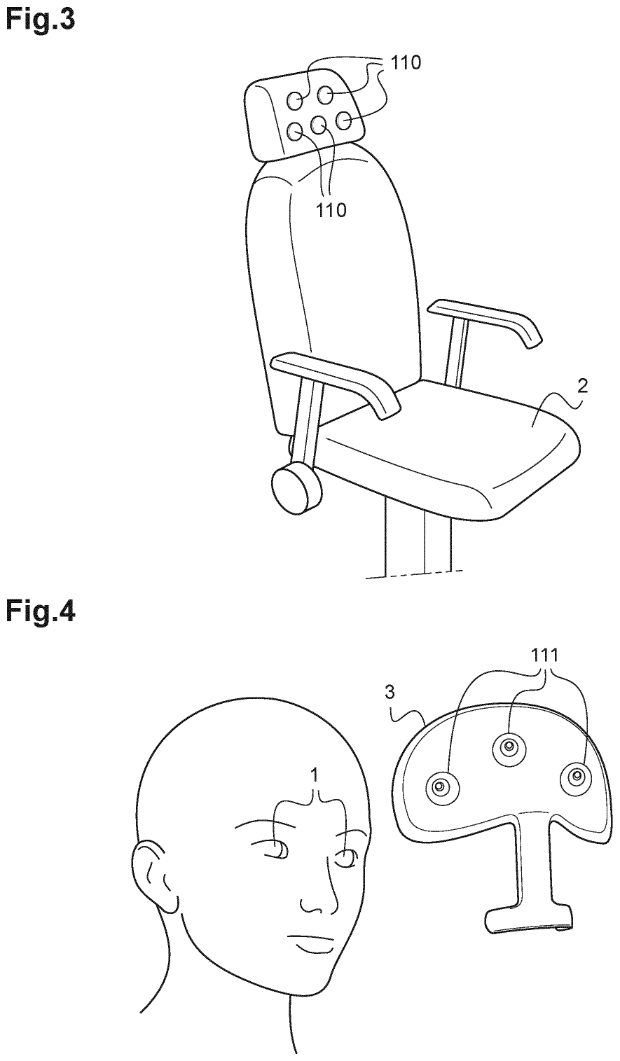

FIG. 3 is a schematic representation of a first embodiment of the neuro-sensor of the device according to the invention,

FIG. 4 is a schematic representation of additional electrodes included in the neuro-sensor of the device of the invention,

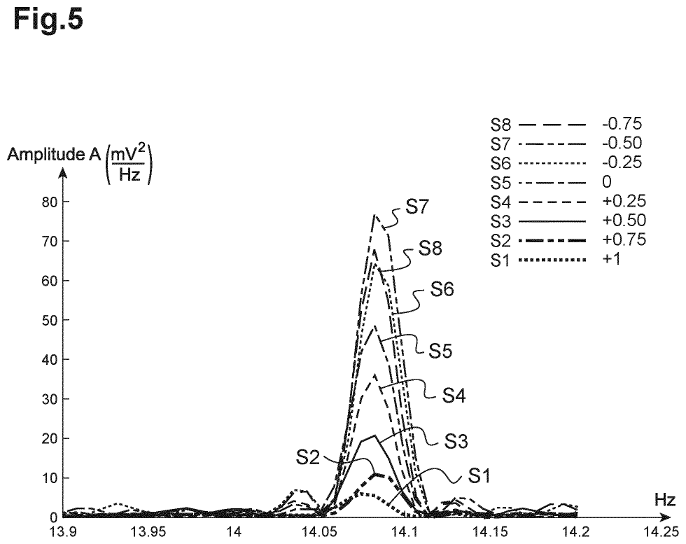

FIG. 5 is a graphic showing eight spectral signals S1 to S8 derived from eight neural signals recorded for a given tested subject, for eight distinct lens powers ranging from +1 diopter to −0.75 diopter, wherein the x-axis gives the frequency in Hertz (Hz) and the y-axis gives the power of the Fourier transformation, referred to as “amplitude” in (mV)²·Hz⁻¹,

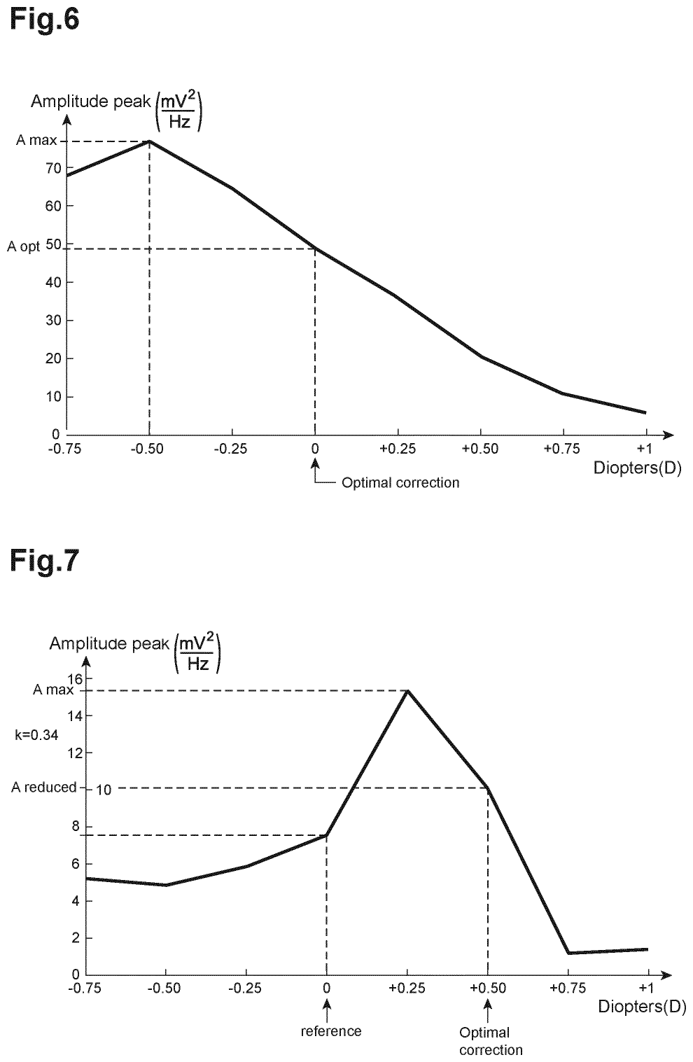

FIG. 6 is a graphic showing the amplitude of the main peak of each spectral signal of FIG. 5 as a function of the lens power (in Diopters),

FIG. 7 is a graphic showing the amplitude of the main peak of each spectral signal obtained from various neural signals recorded for another tested subject, for lens powers ranging from +1 D to −0.75 D, and

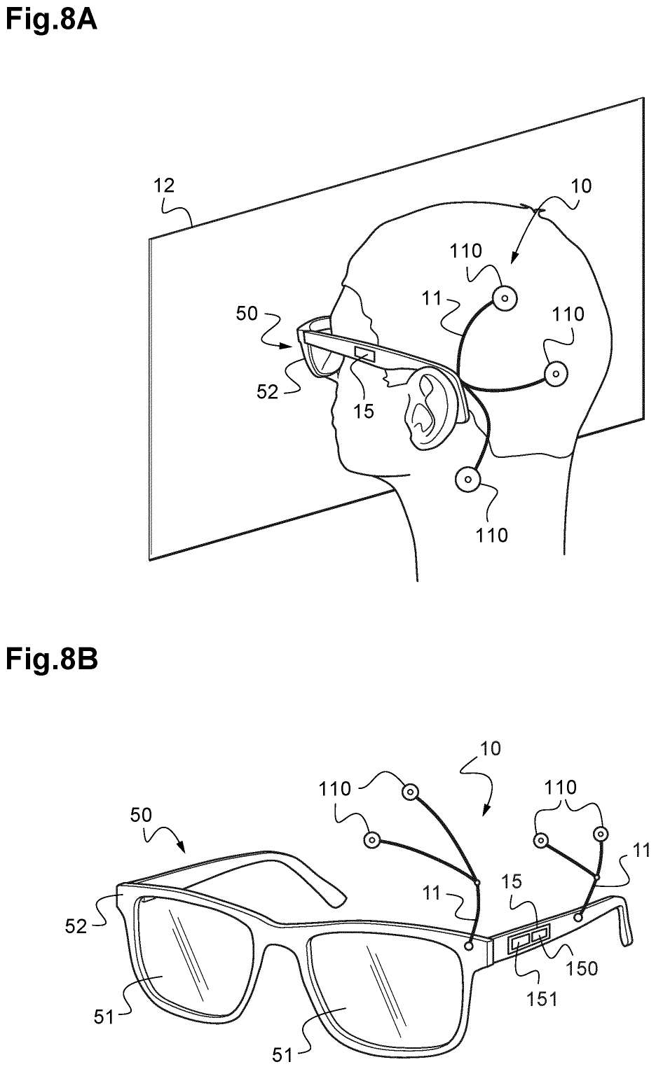

FIGS. 8A and 8B are two examples of an eyeglass that includes active power lenses according to the present disclosure.

The present invention provides a device 10 and a method for objectively determining the optimal correction of an ophthalmic refraction of a subject.

As explained in the introduction, the ophthalmic refraction is the refraction that occurs when a light ray hits one eye of a subject. Such ophthalmic refraction will be called “refraction” in the rest of the description. The refraction is considered to be defective if the image caused by such refraction is formed outside of the retina. In case of defective refraction, it is necessary to know how to correct it in order to improve the visual acuity of the subject. The optimal correction of the refraction is considered to be the correction which allows the subject to have a clear vision in the far distance (also called infinite distance), without accommodating the corresponding eye, or with as little accommodation as possible and with as much comfort as possible. More precisely, the device 10 and the method of invention objectively determine the optimal correction of the (defective) refraction of a subject (called tested subject), based on:
the analysis of respective neural signals of the tested subject that are recorded while at least one of the eyes 1 of the tested subject receives a given visual stimulus through respective lens powers, and
the knowledge of the existence of possible neural artifacts induced by the start of accommodation response of the eye receiving a visual stimulus.

All the features described in relation to the device of the invention, in particular those described in relation with the processor of the device, also apply to the method of the invention, and vice versa.

As shown on FIG. 2, the device 10 of the invention comprises:
at least one neuro-sensor 11 for detecting a neural signal originating from at least one area of the brain of the subject, said neural signal being linked to the visual acuity of the subject of the subject, and
a control unit 15 adapted to:
a) record successive neural signals of the tested subject while an eye 1 of the tested subject receives a visual stimulus through successive distinct lens powers, until one of the recorded neural signal shows a maximum neural activity as compared to all the other recorded neural signals,
b) determine that the optimal correction of the ophthalmic refraction of the subject corresponds to a lens power with which the subject exhibits a neural signal showing a given reduced neural activity as compared to the maximum neural activity obtained in step a).

The device 10 is therefore able to record and analyze neural signals while a visual stimulus is shown to the tested subject through successive lenses L1, L2, L3, L4, L5 of different powers which more or less blur the vision of the tested subject. Based on this analysis, and knowing that part of the neural activity can be induced by accommodation, the device 10 then finds what would be the lens power that would allow the tested subject to see the most clearly the visual stimulus in the far distance with no accommodation or as little accommodation as possible, and as much comfort as possible in the long term. It is to be noted that in the present description, the wording “blur” is opposed to the wording “clear”.

Generally, the visual stimulus is displayed on a screen 12 that the tested subject is asked to look at with one (monocular examination) or both eyes 1 (binocular examination). Preferably, the examination is a monocular examination, the other eye being covered while the first eye 1 is being examined. In case the binocular examination is chosen, both eyes of the tested subject may not necessarily see the visual stimulus through the same lens power.

Classically, the screen 12 is positioned at a distance of approximately six meters (m) of the tested subject to simulate a vision in the far distance. The screen 12 may be included in the device 10 of the invention, in which case the visual stimulus shown on it may be controlled by the control unit 15. In alternative, the screen may be an independent element, excluded from the device 10.

The visual stimulus that the tested subject is asked to look at can be an image of any type. For example, the visual stimulus is a flickering Gabor patch. Such specific visual stimulus allows improving the signal to noise ratio in the neural signal that is recorded by the neuro-sensor 11 of the device 10 and therefore eases the further analysis of each recorded neural signal.

In order to easily change the lens power through which the tested subject sees the visual stimulus, a refractometer 13 (also called “phoropter” or “refractor”) is used.

More precisely, the refractometer 13 is able to place in front of the examined eye 1 of the tested subject a lens L1, L2, L3, L4, L5 of a chosen power. The power of each lens L1, L2, L3, L4, L5 more or less blurs the vision of the eye 1 of the tested subject. The power of the lens L1, L2, L3, L4, L5 is generally given in diopters. In the present description, the power of each tested lens is a relative power as compared to the power of a reference lens LR. The power of the reference lens LR is preferably a rough estimate of the optimal correction of the tested subject. Such rough estimate is for instance obtained by an objective refractive examination as the one described in the introduction. As an alternative, the power of the reference lens can be of 0 diopter (D). As an alternative, the reference power can be the previous known correction of the subject.

The lens power that is tested in front of the eye 1 of the tested subject thus exhibits a positive or a negative diopter as compared to the reference lens power. The more positive is the power, the more it blurs the vision of the corresponding eye 1 of the tested subject.

Preferably, the refractometer 13 is included in the device 10 of the invention. More preferably, the refractometer 13 is controlled by the control unit 15.

Moreover, advantageously, in the device 10, the refractometer 13 is an automated refractometer 13 controlled by the control unit 15 of the device 10. The control unit 15 controls the refractometer 10 to automatically change the power of the lens L1, L2, L3, L4, L5 through which the eye 1 of the tested subject receives the visual stimulus. Such automatic change of the lens L1, L2, L3, L4, L5 placed in front of the eye 1 of the tested subject is preferably based on the analysis of the neural activity of the neural signals previously recorded by the neuro-sensor 11. The change power can be implemented by an operator.

Most preferably, the change of the lens power is implemented by a brain-computer interface, based on the analysis of the neural signals previously recorded for the previously tested lens powers. The tested subject is therefore not asked any question and is not influenced by any operator that would control the change of a lens power by the refractometer 13. Moreover, the change of lens power occurs at the best suited moment for the tested subject. In the present case, the power of the next lens is chosen based on the directly previously recorded and analyzed neural signal, in order for the examination to be efficient.

Notably, if the neural activity of the neural signal recorded for the power tested at time t is higher than the neural activity of the neural signal recorded for the power tested at time t−1, the next power to be tested at time t+1 should be smaller than the power tested at time t. On the contrary, if the neural activity of the neural signal recorded for the power tested at time t is smaller than the neural activity of the neural signal recorded for the power tested at time t−1, the next power to be tested at time t+1 should be greater than the power tested at time t.

The neuro-sensor 11 of the device 10 aims at detecting the neural signals generated in the brain of the tested subject while he tries to see as clearly as possible the visual stimulus shown to him through the lens L1, L2, L3, L4, L5.

In the present example, as shown on FIG. 2, the neuro-sensor 11 comprises at least three electrodes 110, preferably five electrodes 110, that are able to detect the electrical activity of specific groups of neurons that are stimulated when at least one eye of the tested subject tries to clearly see the visual stimulus received in his eye. These specific groups of neurons are preferably located in the occipital area of the brain of the subject. The neural signal detected by these electrodes 110 is linked to the visual acuity of the eye(s) of the tested subject, that is to say that it is linked to the quality of the visual perception of the subject.

The electrodes 110 are designed to be positioned on the back of the head of the tested subject in order to record electroencephalograms (or neural signals) originating from the occipital area of the brain of the tested subject. In the present example, the electrodes 110 are advantageously embedded on the head set of an arm chair 2 (see FIG. 3), on which the tested subject is supposed to sit to proceed to the refractive examination.

As shown on FIGS. 2 and 4, the neuro-sensor 11 may in addition comprise at least 3 additional electrodes 111 that are able to detect the electrical activity of specific groups of neurons that are stimulated when the tested subject tries to accommodate his eye 1 to see clearly the visual stimulus and that may also be involved in image quality processing, judgment and analysis. These specific groups of neurons are for instance located in the prefrontal area of the brain of the subject.

The additional electrodes 111 are frontal electrodes 111 designed to be positioned on the forehead of the tested subject in order to record encephalograms (or neural signals) originating from the prefrontal area of the brain of the tested subject.

As shown on FIG. 4, in the present example, the additional electrodes 111 are for instance embedded on a chin rest or on the frontal support 3 of the refractometer 13.

In the present example, the electrodes 110, and eventual additional electrodes 111, continuously detect the neural activity from the scalp and send it to an amplifier (not represented) that increases the signal in order to ease its analysis.

The recording of each neural signal by the neuro-sensor 11 is preferably performed shortly before, during and following the reception of the visual stimulus by the eye 1 of the tested subject in order to detect the activation of the specific neurons and their relaxation.

The control unit 15 of the device 10 of the invention is configured to communicate with the neuro-sensor 11, and, when appropriate with the refractometer 13 and/or the screen 12. This communication may be established either with wireless communication means or with line-based communication means.

The control unit 15 may be positioned at distance from the tested subject. In an alternative embodiment, the control unit could be worn by the tested subject. In the example shown on the figures, the control unit 15 is placed at distance from the tested subject. In the examples shown on FIGS. 8A and 8B the control unit 15 is worn by the subject. In this last example, the control unit 15 is embedded in eyeglass 50.

As shown on FIG. 2, the control unit 15 here comprises a memory 150 and a processor 151 that communicate with one another as well as with said neuro-sensor 11. The memory 150 and the processor 151 also communicate, when appropriate, with the refractometer 13 and/or with the screen 12. The control unit 15 is for instance integrated in a computer.

In particular, the memory 150 may be configured to receive each neural signal detected by the neuro-sensor 11 and to record (or store) it in order for the processor 151 to analyze each recorded neural signal.

The memory 150 may also be configured to store, in correspondence with each neural signal, which lens power induced said neural signal. The memory 150 can also store the reference lens power of the tested subject.

The analysis of each neural signal recorded in the memory 150 is implemented by the processor 151 of the control unit 15. Such analysis mainly comprises:
the treatment of each neural signal in order to deduce the corresponding neural activity (step a),
the deducing of the maximum neural activity that was detected among all recorded neural signals (step a), and
the determination of the given reduced neural activity that is sought as compared to the maximum neural activity in order to find the lens power that would lead to such reduced neural activity and that therefore corresponds to the optimal correction of the refraction of the tested subject (step b).

Two elements are particularly critical in the invention: the determination of the maximum neural activity, and the determination of the given reduced neural activity.

In practice the reduced neural activity corresponds to the maximum neural activity from which are deduced or removed the neural artifacts induced by accommodation. The accommodation response of the eye inducing neural artifacts is, for instance linked to miosis, convergence or power change in the crystalline lens.

The treatment of each neural signal aims at deducing the neural activity of the tested subject associated with the tested lens power that leads to said neural signal. It is to be noted that, in the area of the brain of the subject associated with visual acuity, the neural activity all the more increases when the subject experiences a better (clearer) perception of the visual stimulus.

In the present example, the neural activity of the tested subject, associated with one lens power, is determined by extracting at least one feature of the neural signal recorded for said lens power.

Here, the feature of the neural signal that gives an indication of the neural activity is the amplitude in the spectral signal derived from the recorded neural signal. A higher neural activity matches with higher amplitude in the spectral signal. More specifically, the neural activity is deduced from the value of the amplitude of the main peak exhibited in the spectral signal.

The analysis of the neural signal, implemented by the processor 151 of the control unit 15 therefore mainly comprises the cleaning of the neural signal from noises and its transformation by a Fourier transformation in order to obtain a spectral signal. In the present case, the feature that is observed is the power of the Fourier transformation, given in square millivolts per Hertz (mV²·Hz⁻¹) as shown on FIG. 5. The power of the Fourier transformation is hereafter called “Amplitude” for simplicity. From the spectral signal can be deduced the amplitude of the main peak, such amplitude being therefore considered as the neural activity of the tested subject. In practice, it is well known how to clean a neural signal from noises and how to transform it into a spectral signal.

In a further aspect of the invention, the device 10 comprises at least one active power lens 51 whose power is driven by the control unit 15. The lens power of the at least one active power lens 51 is arranged and/or configured to be modified when the subject receives the visual stimulus during the step of determining. In addition, the lens power of the at least one active power lens 51 is arranged and/or configured to be equal to the optimal correction once the control unit 15 has determined this value.

In the example of FIGS. 8A and 8B, the at least one active power lens 51 is embedded in an eyeglass 50 intended to be worn by a subject.

In this example, such eyeglass 50 comprises two active power lenses 51 each comprising a lens power arranged and/or configured to be modified by the control unit 15.

The eyeglass 50 further comprises a frame 52, which holds the two active power lenses 51.

The active power lenses 51 are each embedded on a glass of the eyeglass 50.

According to this embodiment, the neural activity sensor 11 may be included in eyewear temple of the frame 52 or directly in contact with the skull of the subject. For this purpose, the device 10 can comprise the electrodes 110.

On FIGS. 8A, 8B, the electrodes 110 of the device 10 are deployed from the eyeglass 50 in order to extend in the areas of the head that will give access to the neural signal that originates from the at least one area of the brain of the subject. For example, the electrodes 110 of the device 10 comprised in the eyeglass 50 are arranged and/or configured to record neural signals originating from the occipital area of the brain of the subject. The device 10 is for example embedded in the frame 52 of the eyeglass 50, or in the vicinity of the portion of frame 52 that ends behind the ear of the subject.

According to this embodiment, the device 10 does not need to be combined with a refractometer 13. The refractometer 13 is directly replaced with the eyeglass 50, which will be able to change the lens power of each active power lens 51 via the control unit 15, in a similar manner as the refractometer 13.

The control unit 15 of the device 10 is configured to communicate with the screen 12. For example, the screen 12 displaying the visual stimulus can be a computer screen or a smartphone screen positioned at a distance of 2 meters or 4 meters from the eyeglass 50.

The lens power of each power active lens 51 of the eyeglass 50 can be modified. For this purpose, the control unit 15, the memory 150, the processor of the device 10 embedded in the eyeglass 50 work as the example of FIG. 2, which uses a refractometer 13.

The control unit 15 is configured to change the lens power of each lens 51 according to the method of the present disclosure. In practice, the control unit 15 of the device 10 controls the lens power of each active power lens 51 through which the eye 1 of the tested subjected receives the visual stimulus. Such change of the power lens is preferably based on the analysis of the neural activity of the signals that have been recorded by the neuro-sensor 11.

The analysis of each neural signal is implemented by the processor of the control unit 15 embedded in the eyeglass 50. Once the optimal correction of the user was determined, the eyeglass 50 stores the results in the memory 150 and uses it to correct the lens power of each active power lens 51 accordingly.

Thus, no operator is needed to change the lens power of the active power lenses 51. The refraction measurements can also be directly performed by the subject without any help of an operator. The device is thus more autonomous than the device 10 disclosed in FIG. 2 in which the help of an operator is needed.

On FIG. 8B, the eyeglass 50 comprises only one control unit 15, which is configured and/or arrange to control the change of the lens power of each active power lens 51 and to record the neural signal. In another embodiment, the eyeglass 50 comprises a control unit 12 associated to each active power lens 51.

The method carried out by the device 10 can be programmed to be performed at a specific date, for example every month, or yearly, monthly, etc and/or can be based on the changes of the correction of the subject, for example in case of myopia evolution, or in case of presbyopia of the subject. When the optimal correction is determined by the control unit 15, the lens power of each lens 51 is changed to be equal to the optimal correction previously determined.

The Eyeglass 50 can comprise different types of active power lenses 51, such as:
Alvarez lenses known from the art,
fluidic lenses, for example as those disclosed in the document EP2149537,
liquid crystal based active lenses, for example as those disclosed in the document EP3115436 using a blue phase state etc.

Each power active lens 51 can be an active lens on an eyewear.

In another embodiment, the active power lens 51 is embedded in a contact lens, for example as disclosed in the document US2020064658. Thus, in this last embodiment, the eyeglass 50 does not comprise any frame 52. The active power lenses 51 are directly hold by the contact lenses on the eyes of the subject. The neural activity sensor 11 may be included in the contact lens. In addition, the sensor may comprise electrodes 110, which may be wireless connected to the active power lenses 51. The control unit 15 is preferably directly comprised in each contact lenses.

In another embodiment, the active power lenses 51 can be active Intraocular Lens Optics (IOL).

As each active power lens 51 is associated to an eye of the subject, the lens power of each active power lens 51 can varied similarly and independently.

On FIG. 5 are shown eight spectral signals derived from neural signals recorded by the electrodes 110, for a tested subject looking at a flickering Gabor patch through respective lenses of various powers (eight different lenses in total). Spectral signal S1 is obtained for a lens power of +1 diopters, spectral signal S2 is obtained for a lens power of +0.75 diopters, spectral signal S3 is obtained for a lens power of +0.5 diopters, spectral signal S4 is obtained for a lens power of +0.25 diopters, spectral signal S5 is obtained for a lens power of 0 diopters, spectral signal S6 is obtained for a lens power of −0.25 diopters, spectral signal S7 is obtained for a lens power of −0.5 diopters and spectral signal S8 is obtained for a lens power of −0.75 diopters. In the present example, the lens power of 0 diopter indicates that the tested subject is viewing the visual stimulus through a lens which power corresponds to the optimal correction of his refraction obtained by a subjective refractive examination. All the other powers given are relative powers as compared to the lens power of the optimal correction.

On FIG. 5, the shape of each spectral signal is similar: each spectral signal comprises a main peak centered on a frequency comprised within the range [5 Hz, 25 Hz], preferably [14 Hz; 15 Hz], said frequency being eventually different for each spectral signal.

The control unit 15 is able to compare all the spectral signals and to find out which spectral signal among S1 to S8 exhibits the peak with the maximum amplitude A.

Here, the lens power that gives the smallest amplitude of the peak of the spectral signal is the lens through which the tested subject perceives the blurriest visual stimulus.

As shown on FIG. 5, the more the lens power decreases, the better is the perception of the visual stimulus by the subject and therefore the greater is the amplitude of the peak of the corresponding spectral signal, up to a certain lens power that gives the highest (or maximum) amplitude of the peak of the spectral signal. Such maximum amplitude corresponds to the perception of the clearest visual stimulus for the brain of the tested subject. However such clearest perception is not comfortable in the long term for the subject as it implies accommodation.

As shown on FIG. 5, when the lens power decreases below the lens power with which the maximum amplitude is obtained, the perception of the visual stimulus by the tested subject degrades and becomes blurry again, and therefore the amplitude of the peak of the corresponding spectral signal decreases. Such phenomenon is due for instance to the fact that the tested subject is not able to accommodate sufficiently with this low lens power, or due to other ophthalmic constraints.

In the example of FIG. 5, the spectral signal exhibiting the main peak with minimum amplitude is the spectral signal S1 obtained for the lens power of +1 diopter, and the spectral signal exhibiting the main peak with maximum amplitude is the spectral signal S7 obtained for the lens power of −0.5 diopter. The spectral signals S2 to S6 obtained with progressively decreasing lens powers, between +0.75 D and −0.25 D, exhibit progressively increasing amplitude of their respective main peak, while the spectral signal S8 obtained for the lens power of −0.75 D exhibits a main peak with smaller amplitude than the maximum amplitude.

As shown on FIG. 5, the amplitude of the spectral signal S5 obtained from the neural signal recorded for the optimal correction (0 D) is not the maximal amplitude that is obtained for the spectral signal S7.

FIG. 6 is obtained after analysis of the spectral signals of FIG. 5. FIG. 6 gives the value of the amplitude A of the main peak of each spectral signal of FIG. 5 as a function of the power (in diopters or D) of the lens that leads to said spectral signal.

FIG. 7 is obtained in a similar way as FIG. 6, but for another tested subject, whose optimal correction is sought. In other words, FIG. 7 is obtained after the neural signals of said other tested subject were recorded while said other tested subject was receiving a visual stimulus (such as a Gabor patch) in his eye, through various lens powers ranging from −0.75 D to +1 D. In FIG. 7, the lens power of 0 D indicates that the tested subject is viewing the visual stimulus through his reference lens which power corresponds to a rough estimate of his optimal correction, obtained for instance from an objective refractive examination as described in the introduction. All the other powers given are relative powers as compared to the reference lens power.

FIGS. 6 and 7 are respectively useful to graphically determine which lens power leads to the neural signal with the maximum neural activity for the respective tested subject, the maximum neural activity being here considered as the maximum amplitude in all the spectral signals.

In the present invention, “a maximum” is defined as being higher than two surrounding points. In other words, the neural signal recorded for a given lens power exhibits a maximum neural activity only if it shows more neural activity than the neural signals recorded for both smaller and greater lens powers surrounding the given lens power.

Of course, the control unit 15 does not need to graphically determine the maximum, and can do so only based on calculations. FIGS. 6 and 7 visually help the reader to understand the evolution of the neural activity with the change of lens power and to understand the determination of said maximum neural activity.

Once the maximum neural activity is found, the processor 151 of the control unit 15 determines the reduced neural activity that should correspond to the perception of the visual stimulus with as little accommodation as possible, or with no accommodation at all. In other words, it determines the reduced neural activity that should be obtained without the accommodation response of the eye. In other words again, it determines that the reduced neural activity corresponds to the maximum neural activity from which is eliminated the neural activity induced by the neural artifacts related to said accommodation response of the eye.

In a first advantageous embodiment of the device, the determination of the reduced neural activity is based on a correction of the maximum neural activity based on a predetermined factor k that is associated with the accommodation response of the eye. Factor k is here associated with the accommodation that induces the neural artifacts of the tested subject. In other words factor k corrects for the additional neural activity induced by neural artifacts linked with accommodation.

Factor k is determined prior to the implementation of the method of the invention. For instance factor k is stored in the memory 150 of the control unit 15.

Factor k is preferably a constant value, lower than or equal to 1. Preferably, the value of factor k is strictly lower than 1.

The value of factor k depends on the feature(s) of the neural signal on which is based the determination of the neural activity. In particular, when the feature of the neural signal that indicates the neural activity is the amplitude A of the peak of the spectral signal, factor k is a constant value chosen between 0.2 and 0.95, preferably between 0.6 and 0.95, more preferably between 0.7 and 0.9. Factor k can notably be equal to 0.2, 0.25, 0.3, 0.35, 0.4, 0.45, 0.5, 0.55, 0.6, 0.65, 0.7, 0.75, 0.8, 0.85, 0.9 or 0.95.

In a first alternative, k is fixed across all individuals.

In a second alternative, factor k may vary from one segment of the population to another. The segments of population could be based on ages for instance: factor k for a tested subject who is a child is different from factor k for a tested subject who is a young adult and different again for a tested subject who is a senior. The segments of population could, in complement or in alternative be based on different eye defect of the subjects, such as ametropia.

Since the maximum neural activity is obtained when accommodation is involved, the value of factor k is all the more closer to 1 when accommodation capacity gets close to zero. In other words, factor k should be greater when the subject has little or no capacity of accommodation remaining rather than when the subject exhibits a great capacity of accommodation.

In practice, in order to determine the value of factor k that will compensate the neural artifacts of the subject associated with the start of accommodation response of the eye, the following steps can be implemented:
c1) a group of subjects whose optimal correction is known is selected;
c2) for each subject,
   successive neural signals of the subject are recorded while at least one eye of the subject receives a visual stimulus through successive distinct lens powers, including the lens power of the optimal correction,
   the neural activity of each recorded neural signal is analyzed, from which is deduced the recorded neural signal that shows the maximum neural activity, and
   the maximum neural activity is compared to the neural activity of the neural signal recorded for the lens power of the optimal correction; and,
c3) factor k is deduced from the step of comparison of neural activities implemented for each subject.

More precisely, at step c1), the group may comprise at least 10 subjects, preferably 20 subjects or more. Of course, the group of subjects preferably does not include the tested subject.

In step c1), the real and precise optimal correction of each eye of each subject of the group is already known, for instance from the subjective refractive examination described in the introduction.

In step c1), the subjects of the group are preferably selected randomly if factor k is fixed across all individuals.

On the contrary, in step c1), the subjects of the group are preferably selected based on features they have in common if factor k varies from one segment of the population to another. In other words, the subjects of the group are selected to be representative of the segment of the population in question. For instance, the subjects may all have the same age, in a given range: they may all have between 5 and 15 years old, or between 15 and 25 years old, or between 25 and 40 years old and so on. In alternative or in complement, the subjects are selected to be part of the same group if they have the same reference lens power, or the same optimal correction. In alternative or in complement, they are selected to be part of the same group if they all have the same defects in the eyes.

At step c2), each subject of the group is tested individually in order to record the neural signal he generates when he receives a visual stimulus through different lenses of different powers, including the power of the optimal correction of said subject.

The neural signal that shows the maximum neural activity is then determined.

In step c3), the maximum neural activity is then compared to the neural activity obtained for the lens power of the optimal correction, from which is deduced factor k.

More precisely, in a first alternative of steps c2) and c3), the determination of the maximum neural activity is done in a similar way as what is explained here above in reference to FIGS. 5 and 6. In other words, the analysis of the neural activity comprises a spectral analysis, wherein a higher neural activity matches with higher amplitude of the main peak in the spectral neural signal. In this alternative, the comparison between the maximum neural activity and the neural activity obtained for the optimal correction comprises the comparison of the amplitudes of the corresponding spectral signals.

An individualized factor ki is determined from this comparison, and compensates for the accommodation response of a given subject.

More precisely, the individualized factor ki can be obtained from the following formula

$$k i = \frac{A max - A opt}{A max}$$

where Amax is the maximum amplitude obtained from all the spectral signals, and Aopt is the amplitude of the spectral signal corresponding to the lens of the optimal correction of the subject (see FIG. 6).

Factor k is then itself deduced from the comparison of all individualized factors ki determined for each subject. In other words, factor k is based on said comparison of amplitudes of the spectral signals, implemented for each subject.

For instance, factor k can be the maximum individualized factor ki obtained for the group of subjects. In alternative, factor k can be the average of all the individualized factors ki.

In a second alternative of steps c2) and c3), the operations of analysis, comparison and deduction are implemented by machine learning. More precisely, a machine learning algorithm allows associating to each tested lens power the corresponding neural activity and determines the relation between the maximum neural activity and the neural activity obtained with the optimal correction and deduces from this correlation the factor k. Such an alternative is preferable if a great variability is found in the individualized factor ki obtained when the first alternative is implemented. If so, the recorded neural signals obtained for each subject and for each lens power are provided to the machine learning algorithm that deduces which feature of the neural signal has to be analyzed to find the maximum neural activity and that determines the factor k that allows comparing the neural signal obtained for the lens power corresponding to the optimal correction and the neural signal showing the maximum neural activity. Such machine learning algorithm for instance comprises an artificial neural network trained with lots of data (neural signal and corresponding lens power including the optimal correction) in order for said artificial neural network to find:
1. the features of the neural activity on which should be based the determination and/or classification of the neural activity, and,
2. factor k thanks to which the maximum neural activity can be compared to the neural activity obtained with the optimal correction.

Now that we have explained how factor k can be determined, we will explain how this factor k can be used to determine the reduced neural activity and then deduce the optimal correction of the refraction of the subject.

Such determination is represented graphically on FIG. 7.

In the present example where the neural activity is obtained through the amplitude of the main peak in the spectral signals, the processor 151 applies factor k to the maximum amplitude Amax found from the analysis of all the spectral signals and therefore finds the reduced amplitude A (reduced):

$$A ( reduced ) = k * A max .$$

The optimal correction of the refraction of the tested subject corresponds to the lens power with which the subject would record a neural signal of said reduced neural activity.

It is to be noted that the reduced neural activity can be the neural activity of one of the recorded neural signal or can be that of an extrapolated neural signal.

In the present example, as shown on FIG. 7, the reduced neural activity corresponds to the neural activity of one of the neural signal.

Once the processor 151 has determined the neural signal of the tested subject associated with the maximum neural activity, and calculated the reduced neural activity through factor k, the processor 151 then finds the corresponding power of lens that would result in the neural signal exhibiting such reduced neural activity.

More precisely, in the present example where the neural activity is obtained through the amplitude of the main in the spectral signals, the processor 151 deduces which lens power should give the reduced amplitude of spectral signal A (reduced). The lens power corresponding to the calculated reduced amplitude can be easily read on FIG. 7 for instance, whatever the reduced neural activity is that of an extrapolated neural signal or that of a recorded neural signal.

The obtained lens power is the optimal correction of the tested subject.

As shown on FIG. 7, the optimal correction of the other tested subject here corresponds to the lens which power is +0.5 D (as compared to the reference lens power here indicated by the 0 D). It is to be noted that the optimal correction is necessarily a lens which power is greater than the power of the lens leading to the maximum amplitude. On FIG. 7, it is confirmed that the optimal correction is neither the power of the reference lens that corresponds to the rough estimate of said optimal correction, neither the power of the lens leading to the maximum amplitude in the spectral signal.

FIG. 1 shows the main steps of the method of the invention for objectively determining the optimal correction of the refraction of the tested subject.

More precisely, the method comprises the steps of:
a) providing said eye 1 of the tested subject with successive distinct lens powers and recording the corresponding successive neural signals of the tested subject while said eye 1 of the tested subject receives a visual stimulus through each lens power, until the recorded neural signal corresponding to one of the lens power shows a maximum neural activity as compared to all the other recorded neural signals (blocks E2 to E5), and,
b) determining that the optimal correction of the ophthalmic refraction of the subject is a lens power with which the subject exhibits a neural signal showing a given reduced neural activity as compared to the maximum neural activity of step a) (blocks E6 and E7).

The method can be implemented by the device 10 of the invention, together with a refractometer 13 and a screen 12 for displaying the visual stimulus as described here above.

More precisely, the processor 151 of the control unit 15 may be configured to implement the steps of calculation of the method of the invention described hereafter.

For instance, step a) is implemented by the refractometer 13, eventually controlled by the control unit 15, and the neural signals are recorded in the memory 150 and analyzed by the processor 151. In step b), the neural signal of reduced neural activity is determined by the processor 151 of the control unit 15.

### Step a)

Step a) is necessarily implemented prior to step b).

At step a), the subject is asked to look at the visual stimulus, preferably with one eye only, through different lenses of different powers. The subject should test at least 3 different lenses of different powers (block E2 of FIG. 1).

To do so, a first, a second and a third lens L1, L2, L3 of a respective first, second and third powers are successively placed in front of his eye. The respective neural signals are successively detected, for instance by the neuro-sensor 11, while the tested subject looks at the visual stimulus. Each detected neural signal is recorded, for instance in the memory 150.

As shown on block E3 of FIG. 1, the neural activity of each neural signal is then analyzed in order to find whether a maximum has been reached, taking into account that a point can only be considered a maximum if it is surrounded by two smaller values. For instance, if the three lens powers are such that the power of the first lens L1 is smaller than the power of the second lens L2 that itself is smaller than the power of the third lens L3, a maximum neural activity has been reached only if the neural activity obtained with the second lens L2 is greater that the neural activity obtained with the first lens L1 and greater than the neural activity obtained with the third lens L3.

As explained here above, the neural activity of the subject, associated with one lens power, is determined by extracting at least one feature of the neural signal recorded for said lens power.

Here, the feature of the neural signal that gives an indication of the neural activity is the amplitude in the spectral signal derived from the recorded neural signal. A higher neural activity matches with higher amplitude in the spectral signal. More specifically, the neural activity is deduced from the value of the amplitude of the main peak exhibited in the spectral signal.

In case a maximum has been reached, the method goes on with step b) (blocks E6 and E7 of FIG. 1).

In case no maximum has been reached yet, a further lens is tested, with a power different from the previously tested powers (block E4 of FIG. 1).

The further lens is thus placed in front of the eye of the subject and the corresponding neural signal is detected by the neuro-sensor 11 and recorded for its further analysis.

Again, it is analyzed whether a maximum neural activity has been reached (block E5 of FIG. 1). If so, the method goes on with step b) (blocks E6 and E7). If not, the method goes on with an iteration of blocks E4 and E5, in order to test a further lens exhibiting a new power. The iteration of blocks E4 and E5 is repeated until a maximum neural activity is reached.

Preferably, when providing the eye 1 of the tested subject with lenses (blocks E2 and E4), the step of diopters between two consecutive tested lens powers is comprised within 0.1 and 0.5 D, for instance it is of 0.25 D. In the present invention, “consecutive” is used to compare the power of the lenses and not necessarily the instant at which said lens powers are provided to the tested subject, while the wording “successive” is used to compare the instant at which said lens powers are provided to the tested subject.

Preferably, each consecutive lens power provided to the eye 1 of the tested subject is chosen based on the reference lens power, which, as previously explained, is a rough estimate of the optimal correction of the tested subject. Such reference lens power can be obtained by an objective refractive examination as explained in the introduction. The reference lens power can for instance be determined in a step prior to the implementation of the method (block E1 of FIG. 1). The reference lens power might also be the previous correction of the tested subject.

More precisely, the step of diopters (D) between two consecutive lens powers is preferably smaller when the lens powers are close to the reference lens power than when the lens powers are distant from the reference lens power. For instance, the step between two consecutive lens powers is of 0.5 D when the power is greater or equal to ±1 D as compared to the reference lens power, and of 0.25 or even 0.1 D when the power is smaller than ±1 D as compared to the reference lens power.

Indeed, the neural signals obtained for the lenses exhibiting a power close to the reference lens power are more likely to exhibit a maximum neural activity. Testing powers of lenses that are close to each other therefore increases the precision in the determination of the maximum neural activity.

According to an advantageous feature of the method of the invention, the power of each successive lens that is placed in front of the eye of the subject at blocks E2 and E4, is not chosen randomly, but in a specific order.

More specifically, the lens powers provided to the eye 1 of the tested subject are successively smaller and smaller, the first lens power provided to the eye being one that blurs the vision of the subject. In other words, the first lens power is preferably chosen with a greater diopter than the reference lens power of the subject, for instance +2 or +1 diopters as compared to the reference lens power. In order to analyze whether a maximum neural activity is reached by one of the neural signal, and knowing that the successive lenses placed in front of the eye 1 of the subject exhibit smaller and smaller powers, it is sufficient to compare the neural activity of the last recorded neural signal with the neural activity of the directly previously recorded neural signal. It is thus considered that a maximum neural activity is reached when the directly previously recorded neural signal shows more neural activity than the last recorded neural signal, said directly previously recorded neural signal being the one that shows the maximum neural activity.

According to an advantageous feature of the method of the invention, the power of the next lens placed in front of the eye of the subject is determined by a brain-computer interface, the lens power being automatically changed based on the analysis of the neural activity of the previously recorded neural signal. By “automatically”, it is meant that no one operates the change of lens apart from the machine, and such operation is based on the analysis of the previously recorded neural signals. Advantageously, the analysis and comparison of two successive recorded neural signals is fast enough to be done in real time, that is to say in less than 1 minute, for instance in approximately 30 seconds. The comparison of two successive neural signals and their respective neural activity can be made by plotting the neural activity (here the amplitude of the peak of the spectral signal) as a function of the lens power, as shown on FIG. 6. The comparison of two successive neural signals and their respective neural activity can also be made by calculation.

The refractive examination according to the method of the invention, implemented with the automated refractometer driven by a brain-computer interface can therefore take approximately 10 minutes in global, that is to say much shorter than the a subjective refractive examination that takes about 15 to 20 minutes.

As soon as a maximum neural activity is found, step a) can stop, and step b) begins (blocks E6 and E7 of FIG. 1).

### Step b)

Once the maximum neural activity has been found, the reduced neural activity is determined from said maximum neural activity.

For instance, the given reduced neural activity is determined by correcting the maximum neural activity based on factor k associated with the accommodation response of the eye, as explained here above in relation with the device of the invention.

In other words, the corrective factor k is applied to said maximum neural activity (block E6 of FIG. 1) in order to find the reduced neural activity that would be obtained if the brain did not exhibit any neural artifact due to accommodation. For instance, factor k is provided to the memory 150 of the control unit 15 prior to the implementation of the method.

In practice, factor k is applied to the feature of the neural signal on which the determination of the maximum neural activity is based. Here, factor k is multiplied to the maximum amplitude Amax obtained from the analysis of the spectral signal in order to find the reduced amplitude A (reduced):

$$A ( reduced ) = k * A max$$

Factor k is predetermined in a step prior to step b), preferably prior to the implementation of the method of the invention, in a way that was explained here above in relation with the device.

As explained in the description of the device, factor k is a constant value that depends on the feature of the neural signal on which is based the determination of the neural activity.

For instance, when the feature of the neural signal that indicates the neural activity is the amplitude of the peak of the spectral signal, factor k is a constant value chosen between 0.6 and 0.95, preferably between 0.7 and 0.9. Factor k can notably be equal to 0.6, 0.65, 0.7, 0.75, 0.8, 0.85, 0.9 or 0.95.

Moreover, factor k can depend on the age of the subject and/or on the defects of the eyes of the subject such as ametropia.

The reduced neural activity can be the neural activity of one of the recorded neural signal or can be the neural activity of an extrapolated neural signal.

In other words, the neural signal exhibiting the reduced neural activity (that is here considered to be the neural signal that exhibits the reduced amplitude A reduced in the spectral signal) is either one of the recorded neural signals or is extrapolated from said recorded neural signals. By extrapolated, it is meant that the neural signal is deduced from the recorded neural signal, although it was not recorded. Such extrapolation can be a graphical or a mathematical extrapolation for instance.

It can then be deduced which lens power would give such neural signal (block E7 of FIG. 1) of reduced neural activity, for example based on FIG. 7. Such lens power is the optimal correction of the refraction of the tested subject.

Thanks to the method and the device of the invention, it is therefore possible to objectively identify the best correction for the tested subject, even in cases where the tested subject is uncertain about his perception.

The invention is not limited to what is described here above.

In particular, in a second advantageous embodiment of the method and the device of the invention, the control unit 15 may be adapted to further provide a machine learning algorithm.

Such a machine learning algorithm may notably be used in order to provide, in step b), both the reduced neural activity and the lens power that should result in such reduced neural activity (Blocks E6 and E7 of FIG. 1).

Such second embodiment is particularly interesting when it seems that factor k greatly varies from one individual to another.

A machine learning algorithm is able to process very complex neural signals and then relate said neural signals to the optimal correction and/or the accommodation response of the eye inducing the neural artifacts in the brain of the subject.

More precisely, a machine learning algorithm takes as input a training set of observed data points to “learn” a data structure such as an equation, a set of rules, or some other data structure. This learned data structure or statistical model may then be used to make generalizations about the training set or predictions about new data. As used herein, “statistical model” refers to any learned and/or statistical data structure that establishes or predicts a relationship between two or more data parameters (e.g., inputs and outputs). Although the invention is described below with reference to neural networks, other types of statistical models may be employed in accordance with the present invention. For example, each data point of the training data set may include a set of values that correlate with, or predict, another value in the data point.

Here, the machine learning algorithm is trained with sets of neural signals that were recorded for many subjects whose eyes were provided with successive distinct lens powers and whose optimal correction is already precisely known, for instance because it was determined by a subjective refractive examination.

In the present invention, the machine learning algorithm may be configured to relate the neural activity of each recorded neural signals provided to the machine learning algorithm to the neural activity of the optimal correction of the refraction of the subject. In other words, the input of the machine learning algorithm may be the recorded neural signals for the tested subject, including the neural signal exhibiting a maximum neural activity, and the output may be the neural signal exhibiting a given neural activity that is characteristic of the optimal correction of the subject. Here the given neural activity is determined by the machine learning algorithm as a reduced neural activity as compared to the maximum neural activity. The reduced neural activity is characteristic of the subject seeing the visual stimulus clearly, without accommodation (or as little accommodation as possible).

Said machine learning algorithm of the control unit 15 may be based either on a Long short-term memory (LSTM) technique or a convolutional neural network (CNN).

LSTM technique is part of recurrent neural networks (RNNs). Classical RNNs techniques comprise a network of neural nodes organized in successive layers. Each node (also called neuron) in a given layer is connected one-way to each of the nodes in the next layer. This structure allows previous moments to be taken into account in the neural network, since a first layer for a former moment t−1 is connected to a second layer for a current moment t. This second layer is also connected to a third layer for a subsequent moment t+1, and so on with a plurality of layers. Each signal provided as an input is therefore processed in a temporal way, taking into account the signals provided at former moments.

CNN techniques use the signals as images, not in a temporal way. The plurality of acquired signals is processed at once with all the data acquired during a given test duration. Mathematical image processing operations are then applied to the image obtained with the plurality of acquired signals, e.g. convolution integral, to determine outputs of the machine learning algorithm.

The machine learning algorithm may comprise a guiding model defining determination rules, said guiding model being configured to guide the prediction of the machine learning algorithm. These rules may comprise sub-correlations between the recorded neural signal exhibiting the maximum neural activity and the recorded neural signal obtained for the optimal correction of the subjects. For example, this guiding model may provide that a given variation of a specific characteristic between the neural signal exhibiting the maximum neural activity and the neural signal obtained with the optimal correction has to be correlated to a variation of accommodation (from little accommodation response to no or almost no accommodation) and has therefore to be correlated to the optimal correction of the refraction. In another example, the guiding model may provide that a predetermined combination of variation of specific characteristics between said neural signal exhibiting the maximum neural activity and said neural signal obtained with the optimal correction implies a variation of accommodation (from little accommodation response to no or almost no accommodation) and therefore implies that the optimal correction of the refraction has been found. This guiding model allows easing the correlation made by the machine learning and therefore both reduces the time taken by the correlation and improves its accuracy.

The control unit 15 may use a machine learning algorithm which is already trained, i.e. the neural network of the machine learning algorithm already comprises an equation or a set of rules configured to provide a correlation between a recorded neural signal exhibiting a maximum neural activity and what should be the neural activity of the neural signal characteristic of the optimal correction. Alternatively, the control unit 15 is configured to train the machine algorithm to determine this correlation.

Training of the machine learning algorithm is preferably performed by providing the algorithm with a plurality of recorded neural signals related to a set of initial subjects whose optimal correction is known. By “initial subjects” we mean subjects which participate in the learning of the machine learning algorithm. In other words, initial subjects provide data allowing the machine learning algorithm to correlate the recorded neural signals to each lens power provided to the subject, in particular to relate the maximum neural activity among all recorded neural signals to the reduced neural activity of the neural signal associated with the optimal correction. On the contrary, a “tested subject” refers to a subject for which the determination of the optimal correction is performed on the basis of the machine learning algorithm, i.e. for which a prediction of his optimal correction may be performed based on recorded neural signals.

This training is repeated many times to make the algorithm more accurate. As an example, training the algorithm may imply at least one hundred initial subjects, preferably a thousand initial subjects.

1. A method for objectively determining an optimal correction of an ophthalmic refraction of a subject, comprising:
a) providing an eye of the subject with successive distinct lens powers and recording the corresponding successive neural signals of the subject while said eye of the subject receives a visual stimulus through each lens power, until the recorded neural signal corresponding to one of the lens power shows a maximum neural activity as compared to all the other recorded neural signals, and
b) determining that the optimal correction of ophthalmic refraction of the subject is a lens power with which the subject exhibits a neural signal showing a given reduced neural activity as compared to the maximum neural activity of step a).

2. The method of claim 1, wherein in step b), the given reduced neural activity is determined by correcting the maximum neural activity based on a predetermined factor k associated with a start of accommodation response of said eye of the subject.

3. The method of claim 1, wherein in step a):
the lens powers provided to the eye of the subject are successively smaller and smaller, a first lens power provided to the eye being one that blurs vision of the subject,
the neural activity of the last recorded neural signal is compared to the neural activity of a directly previously recorded neural signal,
step a) stops when the directly previously recorded neural signal shows more neural activity than the last recorded neural signal, said directly previously recorded neural signal being the one that shows the maximum neural activity.

4. The method of claim 1, wherein in step a), each successive lens power provided to said eye of the subject is chosen based on a reference lens power that is a rough estimate of the optimal correction of the subject.

5. The method of claim 4, wherein each lens power is given in diopter (D) and wherein a step of diopters between two consecutive lens powers is smaller when the lens powers are close to the reference lens power than when the lens powers are distant from the reference lens power.

6. The method of claim 1, wherein step a) is implemented by a brain-computer interface, the lens power being automatically changed based on analysis of the neural activity of a previously recorded neural signal.

7. The method of claim 1, wherein in step b) the neural signal showing the reduced neural activity is one of the recorded neural signals or is extrapolated from said recorded neural signals.

8. The method of claim 2, wherein factor k is determined by:
c1) selecting a group of subjects whose optimal correction is known,
c2) for each subject,
recording successive neural signals of the subject while at least one eye of the subject receives a visual stimulus through successive distinct lens powers, including the lens power of the optimal correction,
analyzing the neural activity of each recorded neural signal, and deduce which recorded neural signal shows the maximum neural activity, and
comparing the maximum neural activity with the neural activity of the neural signal recorded for the lens power of the optimal correction, and
c3) deducing factor k from the comparison of neural activity implemented for each subject.

9. The method of claim 8, wherein in steps c2) and c3), operations of analysis, comparison and deduction are implemented by machine learning.

10. The method of claim 1, wherein the neural activity of the subject, associated with one lens power, is determined by extracting at least one feature of the neural signal recorded for said lens power.

11. The method of claim 10, wherein the feature is amplitude in a spectral signal derived from the recorded neural signal, and wherein a higher neural activity matches with higher amplitude in the spectral signal.

12. A device for objectively determining an optimal correction of an ophthalmic refraction of a subject, comprising
at least one neuro-sensor for detecting a neural signal originating from at least one area of a brain of the subject, said neural signal being linked to visual acuity of eyes of the subject,
control circuitry adapted to
a) record successive neural signals of the subject while at least one eye of the subject receives a visual stimulus through successive distinct lens powers, until one of the recorded neural signal shows a maximum neural activity as compared to all the other recorded neural signals, and
b) determine that the optimal correction of the ophthalmic refraction of the subject corresponds to a lens power with which the subject exhibits a neural signal showing a given reduced neural activity as compared to the maximum neural activity obtained in a).

13. The device of claim 12, further comprising an automated refractometer controlled by the control circuitry to automatically change the power of the lens through which the eye of the subject receives the visual stimulus depending on analysis of neural activity of previously recorded neural signal(s).

14. The device of claim 12, wherein the neuro-sensor further comprises at least 3 electrodes positioned on back of a head of the subject and that are embedded into a head rest of a chair in order to record neural signals originating from an occipital area of the brain of the subject.

15. The device of claim 12, wherein the device further comprises at least one active power lens whose power is driven by the control circuitry for providing said distinct lens powers through which the eye of the subject receives the visual stimulus depending on the analysis of neural activity of previously recorded neural signal(s).

16. The method of claim 2, wherein in step a):
the lens powers provided to the eye of the subject are successively smaller and smaller, a first lens power provided to the eye being one that blurs vision of the subject,
the neural activity of the last recorded neural signal is compared to the neural activity of a directly previously recorded neural signal, and
step a) stops when the directly previously recorded neural signal shows more neural activity than the last recorded neural signal, said directly previously recorded neural signal being the one that shows the maximum neural activity.

17. The method of claim 2, wherein in step a), each successive lens power provided to said eye of the subject is chosen based on a reference lens power that is a rough estimate of the optimal correction of the subject.

18. The method of claim 3, wherein in step a), each successive lens power provided to said eye of the subject is chosen based on a reference lens power that is a rough estimate of the optimal correction of the subject.
